(19) United States
(12) Patent Application Publication
(10) Pub. No.: US 2024/0412850 A1
(43) Pub. Date: Dec. 12, 2024

(54) Air Brain

(71) Applicant: ANUTHEP BENJA-ATHON, Manhasset, NY (US)

(72) Inventor: ANUTHEP BENJA-ATHON, Manhasset, NY (US)

(21) Appl. No.: 18/445,226

(22) Filed: Jun. 7, 2023

(51) Int. Cl. G16H 30/40; G16H 30/20

(52) U.S. Cl. CPC G16H 30/40; G16H 30/20

(57) ABSTRACT

Interactive APS coordinates for creating interactive digital scrolls and interactive digital scrolls-aligned interactive relational dominant health and healthcare data transforming health complaints and ailments into said medical or surgical diagnoses-specific interactive chronologically ordered relational health and healthcare data for associating with highlighted and accentuated APS coordinates in and among viewing screens and touchscreen and ambiences for displaying interactive 2D and 3D holograms.

### DESCRIPTION OF THE PREFERRED EMBODIMENTS

The present invention is abiotic intelligence-created networked communication devices for rendering diagnoses of diseases and disorders causing health complaints ailments, pains and sufferings of billions of people, said networked communication devices creating relational databases of tens of thousands of medical and surgical specific diagnoses-specific chronologically ordered relational health and healthcare data relating to and associating with APS coordinates, in and stored among said storage memories.

One of the preferred embodiments of the present invention described infra is created by the abiotic intelligence comprising processor units attaching to said storage memories for storing computer programs including thesaurus software for instructing said processor units for creating and expanding said relational databases stored in and shared among storage memories, said interactive digital scrolls, said APS coordinates, said APS coordinates and said interactive digital display panels, and commercial software for supporting said abiotic intelligence.

Said abiotic intelligence for translating physicians' professional and technical diagnoses and related medical and surgical chronologically ordered health and healthcare data into lay peoples' English and non-English chronologically ordered words, terms, terminologies and speeches relating to diagnoses in and shared among said abiotic intelligence-networked communication devices.

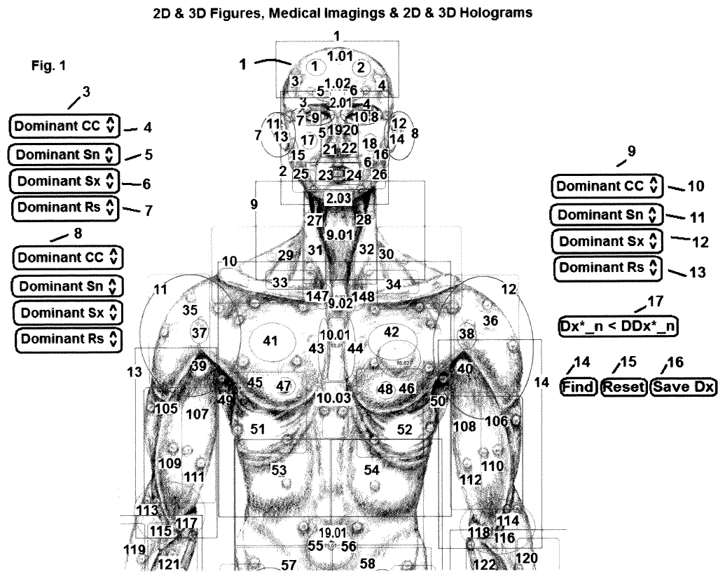

In FIG. 1, Find function 14, Reset function 15 and Save Dx (Diagnosis) function 16 are applicable for said networked communication devices to find the Diagnosis or Diagnoses according to 3) The INTERACTIVE DIGITAL SCROLLS-5) The Transformation; to reset is to restart at 3) The INTERACTIVE DIGITAL SCROLLS; and to Save Dx into said relational databases.

Dx*_n and DDx*_n 17 means diagnoses and related differential diagnoses, respectively, as the products of 3) The INTERACTIVE DIGITAL SCROLLS-5) The Transformation.

Dx*_n and DDx*_n means diagnoses and related differential diagnoses, respectively, as the products of 3) The INTERACTIVE DIGITAL SCROLLS-5) The Transformation.

### 1) The Relational Databases

Said relational databases comprising medical and surgical diagnoses-specific chronologically ordered relational health and healthcare data, and related diagnoses and differential diagnoses, according to said human anatomy, comprising:
said medical and surgical specialties-specific diagnoses and diagnoses-specific relational categories of chief complaints, signs, symptoms, review of systems, laboratory tests, pharmaceuticals, diagnoses, related and linked diagnoses, differential diagnoses, treatments, procedures, devices, equipments, recommendations, referrals, hospitals, clinical notes and messages, drug allergy and food allergy, relating to APS coordinates,
relational dominant chief complaints, relational dominant signs, relational dominant symptoms, relational dominant review of systems and dominant laboratory tests and relational dominant laboratory tests in chronologically ordered words, terms, terminologies, images, videos and speeches, relating to APS coordinates, based on the actual chronologically expressions of diseases and disorders in people, and
relational chief complaints, signs, symptoms, review of systems and laboratory tests in chronologically ordered words, terms, terminologies, images, videos and speeches, relating to APS coordinates, based on the actual chronologically expressions of diseases and disorders in people.

Said networked communication devices creating relational databases comprising medical and surgical diagnoses-specific chronologically ordered relational health and healthcare data relating and associating with APS coordinates.

Said relational databases are ubiquitous and well described in hundreds of medical and surgical textbooks in about 85 medical and surgical specialties such as, but not limited to, An Atlas of Anatomy. Grant J C Boileau. 6th ed. Williams and Wilkins Co 1972. Textbook of Anatomy Hollinshead. Basic Histology. Junqueira I C et al. 2nd ed. Lange Medical Publications 1977. Harrison's Principles of Internal Medicine. Vol 1 & 2, 16th ed. McGraw-Hill 2005. Braunwald's Heart Disease. DP Zipes et al. 11th ed. Elsevier 2019.

### 2) The APS Coordinates

The present invention created said APS coordinate—by the present invention, APS coordinates are incorporated in said relational databases—comprising:

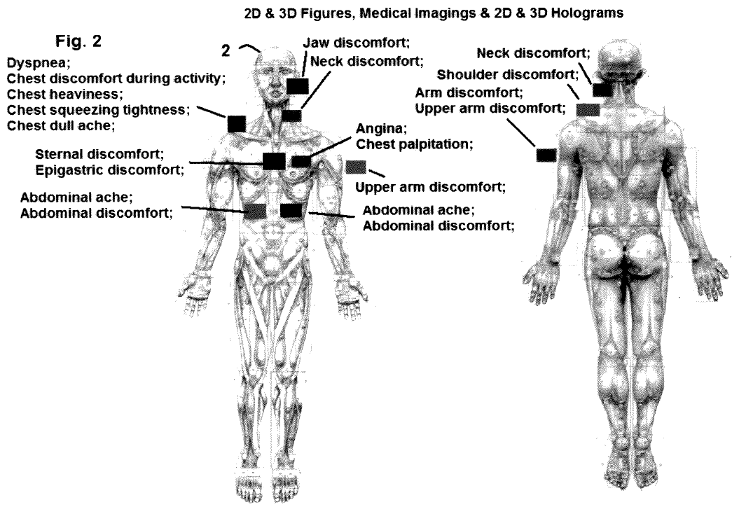

In FIG. 1 and FIG. 2, said interactive APS coordinates comprising interactive and adaptable digital anatomical positioning system of coordinates of 2D & 3D surface, gross and microscopic human anatomy in figures, medical imagings and 2D & 3D holograms-being represented by anterior view 1 and anterior view 2—in and among viewing screens and touchscreens and ambiences for displaying interactive 2D and 3D holograms. Said APS coordinates in said relational databases are represented:
Column 1; Column 2; Column 3; Column 4; comprising:
cc, sn, sx, rs, rx headings; anatomical positionings; anatomical views; cc, sn, sx, rs, rx;
respectively.

In other words, in and among storage memories wherein said relational databases comprising:
Column 1 comprising cc, sn, sx, rs, rx headings;
Column 2 comprising anatomical positionings;
Column 3 comprising anatomical views; and
Column 4 comprising cc, sn, sx, rs, rx;
Said Column 2 is the anatomical positioning system of coordinates being the primary keys, in the relational databases in and shared among storage memories, and in FIG. 1 and FIG. 2 comprising:
numerics in said 2D anterior and posterior surface gross human anatomy;
alphanumerics;
binary codes;
ASCII codes.

The AIR Brain according to claim 1 wherein said APS coordinates, in said relational databases, associating and creating interactive digital scrolls
numerics;
alphanumerics;
binary codes;
ASCII codes.

Said Column 3 is anatomical views applicable to said sample:
Number*; A; means said number in the anterior view
Number*; AM; means said number in the anterior midline
Number*; AS; means said number in the anterior area
Number*; P; means said number in the posterior view
Number*; PM; means said number in the posterior midline
Number*; PS; means said number in the posterior area

In said relational databases, the following is representative relational databases comprising 3 Diagnoses representing about 10,000 Diagnoses and related diagnoses-specific chronologically ordered relational health and healthcare data being created by said networked communication devices and submitted into and shared among said storage memories in said networked communication devices:
Diagnosis 1 Angina Pectoris;
Dominant Chief Complaint; 10.02; A; Angina;
Dominant Sign; 10.02; A; Chest palpitation;
sn; 10; AS; Dyspnea;
Dominant Symptom; AS; Chest discomfort during activity;
sx; 10; AS; Chest heaviness;
sx; 10; AS; Chest squeezing tightness;
sx; 10; AS; Chest dull ache;
sx; 14; AS; Upper arm discomfort;
sx; 28; A; Neck discomfort;
sx; 5; PS; Neck discomfort;
sx; 26; A; Jaw discomfort;
sx; 10.01; AM; Sternal discomfort;
sx; 10.01; AM; Epigastric discomfort;
sx; 16; P; Shoulder discomfort;
sx; 10; PS; Arm discomfort;
sx; 10; PS; Upper arm discomfort;
sx; 51; A; Abdominal ache;
sx; 51; A; Abdominal discomfort;
sx; 52; A; Abdominal ache;
sx; 52; A; Abdominal discomfort;
sx; 1; AS; Emotional stress;
Dominant rs; emotional stress;
Dominant lab; cardiac enzymes;
Diagnosis 2 Unstable Angina;
Dominant Chief Complaint; 10; AS; Acute chest discomfort;
Dominant Sign; 10; AS; Crescendo chest discomfort pattern;
sn; 10.02; A; Angina;
sn; 10; AS; dyspnea at rest;
sn; 10.02; A; S3 S4;
sn; 7; PS; Pulmonary rales;
sn; 10.02; A; Sinus tachycardia;
sn; 13; AS; Hypotension;
sn; 14; AS; Hypotension;
sn; 2; AS; diaphoresis;
sn; 2; AS; pale cool skin;
Dominant Symptom; 10.02; A; New onset severe frank angina;
sx; 10; AS; New onset severe chest discomfort;
sx; 10; AS; Chest discomfort at rest;
sx; 10; A; Chest pain at rest;
sx; 10.02; A; Angina at rest;
sx; 10.02; A; Chest discomfort increasing severity;
sx; 10.02; A; Chest discomfort increasing frequency;
sx; 10.01; AM; substernal pain;
sx; 10.03; AM; epigastric discomfort;
sx; 28; A; Neck discomfort;
sx; 26; A; Jaw discomfort;
sx; 16; P; Shoulder discomfort;
sx; 10; PS; Upper arm discomfort;
Dominant rs; physical exertion;
Dominant lab; cardiac enzymes;
Diagnosis 3 Prinzmetal's Angina;
Dominant Chief Complaint; 10.02; A; Severe ischemic pain at rest;
Dominant Sign; 10.02; A; ST deviation;
sn; 10.02; A; ST depression;
sn; 10.02; A; Coronary artery spasm;
sn; 10.02; A; Acute myocardial infarction;
sn; 10.02; A; Ventricular arrhythmias;
sn; 10.02; A; Heart block;
sn; 10.02; A; Death;
Dominant Symptom; 10; A; Chest discomfort in morning;
sx; 10; A; Chest discomfort at rest;
sx; 10; A; Chest pain at rest;
sx; 10.02; A; Angina at rest;
Dominant rs; resting in bed;
Dominant lab; electrocardiogram;

FIG. 1 and FIG. 2 representing said APS coordinates comprising interactive and adaptable digital anatomical positioning system of coordinates—being said Column 2; Column 3—of 2D & 3D surface, gross and microscopic human anatomy in figures, medical imagings and 2D & 3D holograms, according to human anatomy, in and shared among storage memories and in FIG. 1 and FIG. 2 as 1 in and among said viewing screens and touchscreens of said networked communication devices and ambiences for displaying interactive 2D and 3D holograms.

In FIG. 1 and FIG. 2 representing said APS coordinates comprising interactive and adaptable digital anatomical positioning system of coordinates—being said Column 2; Column 3—of 2D & 3D surface, gross and microscopic human anatomy in figures, medical imagings and 2D & 3D holograms, according to human anatomy, in and shared among storage memories and as 1 in FIG. 1 in and among said viewing screens and touchscreens of said networked communication devices and ambiences for displaying interactive 2D and 3D holograms.

A case in point, a APS coordinate comprising interactive and adaptable digital anatomical positioning system of coordinates 10.02; A; Angina; —being Column 2; Column 3; Column 4; —of 2D & 3D surface, gross and microscopic human anatomy in figures, medical imagings and 2D & 3D holograms, according to human anatomy, in and shared among storage memories and in FIG. 1 and FIG. 2 in and among said viewing screens and touchscreens of said networked communication devices.

In FIG. 1 and FIG. 2, there are a few hundreds red and black digital numerical anatomical positioning system of coordinates (visually obvious in FIG. 1 albeit not all numerics are shown and all numerics are not visually obvious in FIG. 2 due to resolution)—being Column 2; Column 3. Thus, there are thousands of Column 2; Column 3; Column 4 relating to and associating with about 10,000 diagnoses, according to said 1) Relational Databases.

In FIG. 1 and FIG. 2, anterior view 1 and anterior view 2 embodying said APS coordinates coordinating 2D & 3D surface, gross and microscopic human anatomy in figure and 2D & 3D medical imagings such as, but not limited to, people smartphone picture and video imagings (SPI); Magnetic Resonance Imaging (MRI); Pediatric X-ray Imaging; Medical X-Rays; Computed Tomography (CT); Mid-band and High-band Spectrum Imaging; 5G Imaging; Radiography; Mammography; Ultrasound Imaging; Fluoroscopy; and Dental Computed Tomography.

### 3) The Interactive Digital Scrolls

In FIG. 1, said APS coordinates for effecting said networked communication devices for creating and associating with interactive digital scrolls for transforming said health complaints and ailments into said medical or surgical diagnoses-specific interactive chronologically ordered relational health and healthcare data for relating to and associating with interactive highlighted and accentuated APS coordinates, being same APS coordinates as said APS coordinates in said chronologically ordered relational health and healthcare data, in and among viewing screens and touchscreen and ambiences for displaying interactive 2D and 3D holograms.

In FIG. 1, there are a few hundreds groups of interactive digital scrolls-being represented by three representative groups 3, 8 and 9—corresponding to said few hundreds red and black digital numerical anatomical positioning system of coordinates in said APS coordinates.

Individual interactive APS coordinates for effecting said networked communication devices for creating said individual interactive digital scrolls—for example, in FIG. 1, said group 3, group 8 or group 9—comprising scrollable individual relational dominant chief complaints, dominant symptoms, dominant signs and dominant review of systems and dominant laboratory tests, elating and associating with APS coordinates from said relational databases, based on said individual health complaints and ailments.

In detail, in FIG. 1, a networked communication device clicking and selecting interactive APS coordinate 10 representing the chest, based on said health complaints and ailments, whereby, from said relational databases:

said clicked and selected interactive APS coordinate effecting said networked communication device creating an interactive digital scroll 3 and getting all relational dominant chief complaints relating to and associating with said clicked and selected interactive APS coordinate 10, from said relational databases;
In other words, said interactive digital scroll 3 comprising:
interactive digital scroll comprising all interactive scrollable relational Dominant Chief Complaints 4—Acute chest discomfort-relating to and associating with said clicked and selected interactive APS coordinate,
interactive digital scroll comprising all interactive scrollable relational Dominant signs 5 relating to and associating with APS coordinates, which relating to said interactive scrollable relational Dominant Chief Complaints 4,
interactive digital scroll comprising all interactive scrollable relational Dominant symptoms 6 relating to and associating with APS coordinates, which relating to said interactive scrollable relational Dominant Chief Complaints 4,
interactive digital scroll comprising all interactive scrollable relational Dominant review of systems 7—not shown herein—relating to and associating with APS coordinates, which relating to said interactive scrollable relational Dominant Chief Complaints 4, and interactive digital scroll comprising all interactive scrollable relational Dominant laboratory tests not shown herein-relating to and associating with APS coordinates, which relating to said interactive scrollable relational Dominant Chief Complaints 4.

A case in point, for the following Diagnoses, said networked communication device clicking and selecting a interactive APS coordinate 10 creating all interactive scrollable relational Dominant Chief Complaints 4—Acute chest discomfort (among other 10; AS; Dominant Chief Complaints not shown here, from said relational databases. Said 10; AS; Acute chest discomfort effecting said networked communication device getting all relational Dominant signs 5, all relational Dominant symptoms 6, all Dominant review of systems 7 and all Dominant laboratory tests (not shown herein):
Diagnosis 1 Angina Pectoris;
Dominant Chief Complaint; 10; AS; Acute chest discomfort;
Dominant sign; 10.02; A; Angina;
Dominant symptom; 10; AS; Chest discomfort by exertion;
Diagnosis 2 Unstable angina;
Dominant Chief Complaint; 10; AS; Acute chest discomfort;
Dominant sign; 10; AS; Crescendo chest discomfort pattern;
Dominant symptom; 10.02; A; New onset severe frank angina;
Diagnosis 4 Acute myocardial infarction;
Dominant Chief Complaint; 10; AS; Acute chest discomfort;
Dominant sign; 10; AS; Chest pain unrelieved by nitroglycerin;
Dominant symptom; 10.02; A; New onset severe frank angina;
Diagnosis 5 Aortic stenosis;
Dominant Chief Complaint; 10; AS; Acute chest discomfort;
Dominant sign; 10.02; A; Late-peaking systolic murmur;
Dominant symptom; 10; AS; Chest discomfort by exertion;
Diagnosis 6 Aortic dissection;
Dominant Chief Complaint; 10; AS; Acute chest discomfort;
Dominant sign; 111; A; Absent pulses;
Dominant symptom; 10; AS; Anterior chest pain;
Diagnosis 7 Dilated Cardiomyopathy;
Dominant Chief Complaint; 10; AS; Orthorpnea;
Dominant sign; 10.02; A; S3 S4;
Dominant symptom; 10; A; Chest discomfort in morning;
and other relevant diagnoses not shown herein such as, but not limited to, Moderate Congestive Heart Failure; Pericarditis; Hypertension; Pulmonary embolism; Pulmonary hypertension; Pneumonia; Pleuritis; Spontaneous pneumothorax; Esophageal reflux; Esophageal spasm; Peptic ulcer; Gallbladder disease; and Herpes zoster;
Similarly, said one preferred embodiment is applicable to another case in point, a networked communication device clicking and selecting a interactive APS coordinate 10.02 representing the heart, based on said health complaints and ailments, whereby, from said relational databases, and getting all relational dominant chief complaints-Severe ischemic pain at rest, and many more Dominant Chief Complaints (not shown herein); relating to and associating with said clicked and selected interactive APS coordinate 10.02; A; from said relational databases;
Diagnosis 3 Prinzmetal's Angina;
Dominant Chief Complaint; 10.02; A; Severe ischemic pain at rest;
Dominant sign; 10.02; A; ST deviation;
Dominant symptom; 10; A; Chest discomfort in morning;
and other relevant diagnoses whose Dominant Chief Complaint; 10.02; A; are not shown herein.

In other words, interactive digital scroll 9 comprising:
interactive digital scroll comprising all interactive scrollable relational Dominant Chief Complaints 10—Severe ischemic pain at rest—relating to and associating with said clicked and selected interactive APS coordinate,
interactive digital scroll comprising all interactive scrollable relational Dominant signs 11—ST deviation and many more signs-relating to and associating with APS coordinates, which relating to said interactive scrollable relational dominant chief complaints,
interactive digital scroll comprising all interactive scrollable relational Dominant symptoms 12—Chest discomfort in morning and many more symptoms—relating to and associating with APS coordinates, which relating to said interactive scrollable relational dominant chief complaints,
interactive digital scroll comprising all interactive scrollable relational dominant review of systems 13—not shown herein—relating to and associating with APS coordinates, which relating to said interactive scrollable relational dominant chief complaints, and
interactive digital scroll comprising all interactive scrollable relational dominant laboratory tests (not shown in FIG. 1 and FIG. 2 relating to and associating with APS coordinates, which relating to said interactive scrollable relational dominant chief complaints.

Thus, said two truncated representations are applicable to said few hundreds interactive digital scrolls corresponding to said few hundreds red and black digital numerical anatomical positioning system of coordinates in said APS coordinates.

### 4) The Interactive Digital Scrolls-Aligned Relational Dominant Data

Said networked communication devices aligning individual interactive relational dominant chief complaints, individual interactive relational dominant signs, individual interactive relational dominant symptoms, individual interactive relational dominant review of systems and individual interactive relational dominant laboratory tests among said interactive digital scrolls whereby said individual interactive digital scrolls-aligned dominant data comprising one interactive relational dominant chief complaints, one interactive relational dominant sign, one interactive relational dominant symptom, one interactive relational dominant review of system and one interactive relational dominant laboratory test—pathognomonic, similar or approximate in diseases and disorders—also known herein as interactive digital scrolls-aligned relational dominant data, based on said health complaints and ailments.

### 5) The Transformation of Said People's Health Complaints, Ailments, Pains and Sufferings

Said one interactive digital scrolls-aligned dominant relational data effecting said networked communication devices getting said medical or surgical diagnoses-specific chronologically ordered relational health and healthcare data including said one interactive digital scrolls-aligned dominant relational data which relating to and associating with interactive highlighted and accentuated APS coordinates—anterior view 2 in FIG. 2—same as APS coordinates in said medical or surgical diagnoses-specific chronologically ordered relational health and healthcare data and related diagnoses and related differential diagnoses, in and shared among said storage memories, in and among viewing screens and touchscreen and ambiences for displaying interactive 2D and 3D holograms:

In FIG. 1 and FIG. 2, anterior view 1 and anterior view 2 embodying said APS coordinates coordinating 2D & 3D surface, gross and microscopic human anatomy in FIG. 1 and FIG. 2, anterior view 1 and anterior view 2 and 2D & 3D medical imagings such as, but not limited to, people smartphone picture and video imagings (SPI); Magnetic Resonance Imaging (MRI); Pediatric X-ray Imaging; Medical X-Rays; Computed Tomography (CT); Mid-band and High-band Spectrum Imaging; 5G Imaging; Radiography; Mammography; Ultrasound Imaging; Fluoroscopy; and Dental Computed Tomography.

Said networked communication devices for creating interactive digital panels for approving said medical or surgical diagnoses-specific chronologically ordered relational health and healthcare data and diagnoses and related differential diagnoses whereby individual approve functions attaching to and submitting said chronologically ordered relational health and healthcare data into and shared among said storage memories;

Said networked communication devices for creating interactive digital panels for editing said medical or surgical diagnoses-specific chronologically ordered relational health and healthcare data and diagnoses and related differential diagnoses whereby individual approve functions attaching to and submitting said chronologically ordered relational health and healthcare data into and shared among said storage memories;
said networked communication devices for rendering said APS coordinates and relating edited individual chronologically ordered relational health and healthcare data and said diagnoses of said diseases and disorders and related differential diagnoses, as highlighted and accentuated—colors in anterior view 2 in FIG. 2—digital anatomical positioning system of coordinates, being same as said APS coordinates in said chronologically ordered relational health and healthcare data, relating and associating with said edited, of 2D & 3D surface, gross and microscopic human anatomy in figures, medical imagings and said 2D & 3D holograms being electronically and wirelessly shared among said networked communication devices; and
said networked communication devices for rendering said APS coordinates and relating edited individual chronologically ordered relational health and healthcare data and said diagnoses of said diseases and disorders and related differential diagnoses, as highlighted and accentuated—colors in anterior view 2 in FIG. 2—digital anatomical positioning system of coordinates, being same as said APS coordinates in said edited chronologically ordered relational health and healthcare data, relating and associating with said edited, of 2D & 3D surface, gross and microscopic human anatomy in figures, medical imagings and said 2D & 3D holograms being electronically and wirelessly shared among said networked communication devices.

Said interactive highlighted and accentuated APS coordinates—anterior view 2 in FIG. 2—enable said interactive highlighted and accentuated APS coordinates to repeat 2) The APS coordinates-5) The Transformation of Said People's Health Complaints, Ailments, Pains and Sufferings by the same networked communication device and by same networked communication devices-applicable to FIG. 1 and FIG. 2, anterior view 1 and anterior view 2 embodying said APS coordinates coordinating 2D & 3D surface, gross and microscopic human anatomy in figure and 2D & 3D medical imagings such as, but not limited to, people smartphone picture and video imagings (SPI); Magnetic Resonance Imaging (MRI); Pediatric X-ray Imaging; Medical X-Rays; Computed Tomography (CT); Mid-band and High-band Spectrum Imaging; 5G a Imaging; Radiography; Mammography; Ultrasound Imaging; Fluoroscopy; and Dental Computed Tomography.

Said one preferred embodiment is applicable to said few hundreds interactive digital scrolls and thousands of said interactive digital scrolls-aligned dominant relational data corresponding to said few hundreds red and black digital numerical anatomical positioning system of coordinates in said APS coordinates.

1. A system of networked communication devices for rendering diagnoses of diseases and disorders causing health complaints and ailments of billions of people, said networked communication devices for creating relational databases of medical and surgical diagnoses-specific chronologically ordered relational health and healthcare data relating and associating with APS coordinates, said APS coordinates for creating and associating with interactive digital scrolls comprising interactive scrollable lists of interactive relational dominant chief complaints for relating and associating with interactive relational dominant health and healthcare data relating to and associating with APS coordinates, wherein interactive digital scrolls-aligned relational dominant chief complaints and relational dominant health and healthcare data also known as interactive digital scrolls-aligned relational dominant data for transforming said health complaints and ailments into said medical or surgical diagnoses-specific interactive chronologically ordered relational health and healthcare data for relating to and associating with highlighted and accentuated APS coordinates, being same APS coordinates as said APS coordinates in said chronologically ordered relational health and healthcare data, in and among viewing screens and touchscreen and ambiences for displaying interactive 2D and 3D holograms, and in and stored among storage memories, collectively known herein as abiotic intelligence-rendered brain also known as AIR Brain comprising:
said networked communication devices comprising networked servers, mobile communication devices, desktops and holography devices comprising said processor units attaching to said storage memories, and said viewing screens and touchscreen and ambiences for displaying interactive 2D and 3D holograms;
said networked servers, mobile communication devices and desktops for producing and APS coordinating 2D & 3D surface, gross and microscopic human anatomy in figure and 2D & 3D medical imagings;
said abiotic intelligence comprising processor units attaching to said storage memories for storing computer programs including thesaurus software for instructing said processor units for creating and expanding said chronologically ordered health and healthcare data, stored in and shared among storage memories, said interactive digital scrolls, said APS coordinates, said APS coordinates and said interactive digital display panels, and commercial software for supporting said abiotic intelligence;
said abiotic intelligence for translating physicians' professional and technical diagnoses and related medical and surgical chronologically ordered health and healthcare data into lay peoples' English and non-English chronologically ordered words, terms, terminologies and speeches relating to diagnoses in and shared among said abiotic intelligence-networked communication devices;
interactive digital display panels for getting, approving and editing said individual chronologically ordered relational health and healthcare data and said diagnoses of said diseases and disorders and related differential diagnoses as approved or edited individual chronologically ordered relational health and healthcare data in and shared among storage memories, said viewing screens and touchscreens and holography devices for displaying 2D & 3D holograms;
said networked communication devices for rendering said APS coordinates and relating edited individual chronologically ordered relational health and healthcare data and said diagnoses of said diseases and disorders and related differential diagnoses, as interactive highlighted and accentuated-colors in 2 in the Diagram-digital anatomical positioning system of coordinates, being same as said APS coordinates in said edited chronologically ordered relational health and healthcare data, relating and associating with said edited, of 2D & 3D surface, gross and microscopic human anatomy in figures, medical imagings and said 2D & 3D holograms being electronically and wirelessly shared among said networked communication devices.

2. The AIR Brain according to claim 1 wherein said relational databases of medical and surgical diagnoses-specific chronologically ordered relational health and healthcare data, according to said human anatomy, comprising:
said medical and surgical specialties-specific diagnoses and diagnoses-specific relational categories of chief complaints, signs, symptoms, review of systems, laboratory tests, pharmaceuticals, diagnoses, related and linked diagnoses, differential diagnoses, treatments, procedures, devices, equipments, recommendations, referrals, hospitals, clinical notes and messages, drug allergy and food allergy, relating to APS coordinates,
relational dominant chief complaints, relational dominant signs, relational dominant symptoms, relational dominant review of systems and relational dominant laboratory tests and relational dominant laboratory tests in chronologically ordered words, terms, terminologies, images, videos and speeches, relating to APS coordinates, based on the actual chronologically expressions of diseases and disorders in people, and
relational chief complaints, signs, symptoms, review of systems and laboratory tests in chronologically ordered words, terms, terminologies, images, videos and speeches, relating to APS coordinates, based on the actual chronologically expressions of diseases and disorders in people.

3. The AIR Brain according to claim 1 wherein said 2D & 3D medical imagings comprising:
Smartphone Photography Imaging (SPI);
Magnetic Resonance Imaging (MRI);
Pediatric X-ray Imaging;
Medical X-Rays;
Computed Tomography (CT);
Mid-band and High-band Spectrum Imaging;
5G Imaging;
Radiography;
Mammography;
Ultrasound Imaging
Fluoroscopy; and
Dental Computed Tomography.

4. The AIR Brain according to claim 1 wherein said APS coordinates comprising:
interactive and adaptable digital anatomical positioning system of coordinates of 2D & 3D surface, gross and microscopic human anatomy in figures, medical imagings and 2D & 3D holograms in and among viewing screens and touchscreens and ambiences for displaying interactive 2D and 3D holograms;
individual APS coordinates for effecting said networked communication devices for creating said individual interactive digital scrolls comprising scrollable individual relational dominant chief complaints, dominant symptoms, dominant signs and dominant review of systems and dominant laboratory tests, relating and associating with APS coordinates from said relational databases, based on said individual health complaints and ailments.

5. The digital anatomical positioning system of coordinates according to claim 4 in said relational databases comprising:
numerics;
alphanumerics;
binary codes;
ASCII codes.

6. The AIR Brain according to claim 1 wherein said APS coordinates for associating and creating interactive digital scrolls
numerics;
alphanumerics;
binary codes;
ASCII codes.

7. The AIR Brain according to claim 1 wherein said interactive relational dominant health and healthcare data relating to and associating with APS coordinates comprising:
interactive scrollable lists of interactive relational dominant chief complaints relating to and associating with networked communication devices-clicked and -selected interactive APS coordinates effecting said networked communication devices for creating said interactive scrollable lists of interactive relational dominant chief complaints;
interactive scrollable list of interactive relational dominant signs relating to APS coordinates in said relational databases;
interactive scrollable list of interactive relational dominant symptoms relating to APS coordinates in said relational databases;
interactive scrollable list of interactive relational dominant review of systems relating to APS coordinates in said relational databases; and
interactive scrollable list of interactive relational dominant laboratory tests relating to APS coordinates in said relational databases.

8. The AIR Brain according to claim 1 wherein said interactive digital scrolls for creating interactive digital scrolls-aligned relational dominant health and healthcare data comprising:
interactive scrollable list of interactive relational dominant chief complaints relating said relational dominant health and healthcare data comprising:
interactive scrollable list of interactive relational dominant signs relating to said list of interactive relational dominant chief complaints;
interactive scrollable list of interactive relational dominant symptoms, relating to and associating with APS coordinates, relating to said
list of interactive relational dominant chief complaints;
interactive scrollable list of interactive relational dominant review of systems, relating to and associating with APS coordinates, relating to said list of interactive relational dominant chief complaints;
interactive scrollable list of interactive relational dominant laboratory tests, relating to and associating with APS coordinates, relating to said list of interactive relational dominant chief complaints;
said relational dominant chief complaints, relational dominant symptoms, relational dominant signs and relational dominant review of systems and relational dominant laboratory tests relating to APS coordinates in said relational databases; and
said relational dominant chief complaints, relational dominant symptoms, relational dominant signs and relational dominant review of systems and relational dominant laboratory tests relating diagnoses and related differential diagnoses in said relational databases.

9. The AIR Brain according to claim 1 wherein said interactive digital scrolls-aligned relational dominant health and healthcare data comprising individual adaptable alignments of individual relational dominant chief complaints, individual relational dominant signs, individual relational dominant symptoms, individual relational dominant review of system, and individual relational dominant laboratory tests among:
interactive scrollable list of interactive relational dominant chief complaints relating said relational dominant health and healthcare data comprising:
interactive scrollable list of interactive relational dominant signs relating to said list of interactive relational dominant chief complaints;
interactive scrollable list of interactive relational dominant symptoms, relating to and associating with APS coordinates, relating to said list of interactive relational dominant chief complaints;
interactive scrollable list of interactive relational dominant review of systems, relating to and associating with APS coordinates, relating to said list of interactive relational dominant chief complaints;
interactive scrollable list of interactive relational dominant laboratory tests, relating to and associating with APS coordinates, relating to said list of interactive relational dominant chief complaints;

10. The AIR Brain according to claim 1 wherein said interactive digital scrolls-aligned relational dominant data for transforming said health complaints and ailments comprising:
said medical diagnoses-specific chronologically ordered relational health and healthcare data including digital scrolls-aligned relational dominant health and healthcare data in said relational databases; and
said surgical diagnoses-specific chronologically ordered relational health and healthcare data including digital scrolls-aligned relational dominant health and healthcare data in said relational databases.

11. The AIR Brain according to claim 1 wherein said interactive digital display panels comprising:
individual approve functions for attaching to and submitting said chronologically ordered relational health and healthcare data into and shared among said storage memories; and
individual delete functions for attaching to and deleting individual health and healthcare data.

12. A system of networked communication devices for creating APS coordinates for creating and associating with interactive digital scrolls comprising interactive relational dominant health and healthcare data relating and associating with APS coordinates, wherein interactive digital scrolls-aligned individual interactive relational dominant health and healthcare data for transforming said health complaints and ailments into said medical or surgical diagnoses-specific interactive chronologically ordered relational health and healthcare data for relating to and associating with highlighted and accentuated APS coordinates, being same APS coordinates as said APS coordinates in said chronologically ordered relational health and healthcare data, in and among viewing screens and touchscreen and ambiences for displaying interactive 2D and 3D holograms, and in and stored among storage memories, collectively known herein as abiotic intelligence-rendered brain also known as AIR Brain comprising:
said networked communication devices comprising networked servers, mobile communication devices, desktops and holography devices comprising said processor units attaching to said storage memories, and said viewing screens and touchscreen;
said networked servers, mobile communication devices and desktops for producing and APS coordinating 2D & 3D surface, gross and microscopic human anatomy in figure and 2D & 3D medical imagings;
said abiotic intelligence comprising processor units attaching to said storage memories for storing computer programs including thesaurus software for instructing said processor units for creating and expanding said chronologically ordered health and healthcare data, stored in and shared among storage memories, said interactive digital scrolls, said APS coordinates, said APS coordinates and said interactive digital display panels, and commercial software for supporting said abiotic intelligence;
said abiotic intelligence for translating physicians' professional and technical diagnoses and related medical and surgical chronologically ordered health and healthcare data into lay peoples' English and non-English chronologically ordered words, terms, terminologies and speeches relating to diagnoses in and shared among said abiotic intelligence-networked communication devices;
said relational databases of medical and surgical diagnoses-specific chronologically ordered relational health and healthcare data relating and associating with APS coordinates;
said APS coordinates for effecting said networked communication devices for creating said interactive digital scrolls;
said individual interactive digital scrolls;
said interactive digital scrolls-aligned relational data relating to and associating with APS coordinates;
said interactive digital display panels;
said APS coordinates in said approved or edited individual chronologically ordered relational health and healthcare data and said diagnoses of said diseases and disorders and related differential diagnoses;
said APS coordinates relating approved individual chronologically ordered relational health and healthcare data and said diagnoses of said diseases and disorders and related differential diagnoses, as interactive highlighted and accentuated digital anatomical positioning system of coordinates, being same as said APS coordinates in said chronologically ordered relational health and healthcare data, relating and associating with said approved, of 2D & 3D surface, gross and microscopic human anatomy in figures, medical imagings and 2D & 3D holograms; and
said APS coordinates relating edited individual chronologically ordered relational health and healthcare data and said diagnoses of said diseases and disorders and related differential diagnoses, as interactive highlighted and accentuated digital anatomical positioning system of coordinates, being same as said APS coordinates in said chronologically ordered relational health and healthcare data, relating and associating with said edited, of 2D & 3D surface, gross and microscopic human anatomy in figures, medical imagings and 2D & 3D holograms.

13. The AIR Brain according to claim 12 wherein said APS coordinates comprising:
interactive and adaptable digital anatomical positioning system of coordinates of 2D & 3D surface, gross and microscopic human anatomy in figures, medical imagings and 2D & 3D holograms, according to human anatomy, in and among viewing screens and touchscreens and ambiences for displaying interactive 2D and 3D holograms;
individual APS coordinates for effecting said networked communication devices for creating said individual interactive digital scrolls comprising scrollable individual relational dominant chief complaints, dominant symptoms, dominant signs and dominant review of systems and dominant laboratory tests, relating and associating with APS coordinates from said relational databases, based on said individual health complaints and ailments.

14. In AIR Brain, the method of abiotic intelligence-created networked communication devices transforming people's health complaints ailments, pains and sufferings into medical or surgical diagnoses and related chronologically ordered relational health and healthcare data relating to and associating with APS coordinates, in relational databases in and stored among said storage memories and in and among viewing screens and touchscreens and ambiences for displaying interactive 2D and 3D holograms, said networked communication devices clicking individual APS coordinates creating interactive digital scrolls comprising interactive scrollable lists of interactive relational dominant chief complaints relating to and associating with said clicked APS coordinates,
selecting said individual interactive dominant chief complaints and thence individual interactive scrollable lists of interactive relational dominant data relating to said dominant chief complaints, according to said relational databases,
aligning said individual interactive dominant chief complaints and said individual interactive relational dominant data said interactive digital and thence creating individual scrolls-aligned dominant chief complaints and relational dominant data, based on said health complaints ailments, pains and sufferings,
getting and thence relating and associating said interactive digital scrolls-aligned dominant chief complaints and relational dominant data relating to said medical or surgical diagnoses-specific interactive chronologically ordered relational health and healthcare data, relating to and associating with APS coordinates, with highlighted and accentuated same APS coordinates in said chronologically ordered relational health and healthcare data, in and among viewing screens and touchscreen and ambiences for displaying interactive 2D and 3D holograms, and in and stored among storage memories, said method comprising the processes of:
said relational databases creating said networked communication devices which are networked servers, mobile communication devices, desktops and holography devices comprising said processor units attaching to said storage memories, and said viewing screens and touchscreen and ambiences for displaying interactive 2D and 3D holograms;
said networked servers, mobile communication devices and desktops producing and APS coordinating 2D & 3D surface, gross and microscopic human anatomy in figure and 2D & 3D medical imagings;
said abiotic intelligence comprising processor units attaching to said storage memories for storing computer programs including thesaurus software for instructing said processor units for creating and expanding said chronologically ordered health and healthcare data, stored in and shared among storage memories, said interactive digital scrolls, said APS coordinates, said APS coordinates and said interactive digital display panels, and commercial software for supporting said abiotic intelligence;
said abiotic intelligence for translating physicians' professional and technical diagnoses and related medical and surgical chronologically ordered health and healthcare data into lay peoples' English and non-English chronologically ordered words, terms, terminologies and speeches relating to diagnoses in and shared among said abiotic intelligence-networked communication devices;
interactive digital display panels for getting, approving and editing said individual chronologically ordered relational health and healthcare data and said diagnoses of said diseases and disorders and related differential diagnoses as approved or edited interactive individual chronologically ordered relational health and healthcare data in and shared among storage memories, said viewing screens and touchscreens and holography devices for displaying 2D & 3D holograms;
said APS coordinates relating and associating said interactive approved individual chronologically ordered relational health and healthcare data and said diagnoses with same APS coordinates in said chronologically ordered relational health and healthcare data, being highlighted and accentuated, in and among viewing screens and touchscreen and ambiences for displaying interactive 2D and 3D holograms; and
said APS coordinates relating and associating said interactive edited individual chronologically ordered relational health and healthcare data and said diagnoses with same APS coordinates in said chronologically ordered relational health and healthcare data, being highlighted and accentuated, in and among viewing screens and touchscreen and ambiences for displaying interactive 2D and 3D holograms.

15. The method according to claim 14 whereby said interactive digital scrolls transforming said individual health complaints and ailments comprising the processes of said networked communication devices:
clicking and selecting a interactive APS coordinate, based on said individual health complaints and ailments, whereby, from said relational databases:
said clicked and selected interactive APS coordinate effecting said networked communication devices creating interactive digital scrolls and getting all relational dominant chief complaints relating to and associating with said clicked and selected interactive APS coordinate;
creating said interactive digital scrolls comprising:
interactive digital scroll comprising all interactive scrollable relational dominant chief complaints relating to and associating with said clicked and selected interactive APS coordinate,
interactive digital scroll comprising all interactive scrollable relational dominant signs relating to and associating with APS coordinates, which relating to said interactive scrollable relational dominant chief complaints,
interactive digital scroll comprising all interactive scrollable relational dominant symptoms relating to and associating with APS coordinates, which relating to said interactive scrollable relational dominant chief complaints,
interactive digital scroll comprising all interactive scrollable relational dominant review of systems relating to and associating with APS coordinates, which relating to said interactive scrollable relational dominant chief complaints,
interactive digital scroll comprising all interactive scrollable relational dominant laboratory tests relating to and associating with APS coordinates, which relating to said interactive scrollable relational dominant chief complaints;
aligning individual interactive relational dominant chief complaints, individual interactive relational dominant signs, individual interactive relational dominant symptoms, individual interactive relational dominant review of systems and individual interactive relational dominant laboratory tests among said interactive digital scrolls, based on said individual health complaints and ailments; and
said networked communication devices using said aligned relational dominant data relating to APS coordinates to get said medical or surgical diagnoses-specific interactive chronologically ordered relational health and healthcare data for relating to and associating with highlighted and accentuated APS coordinates, being same APS coordinates as said APS coordinates in said chronologically ordered relational health and healthcare data, in and among viewing screens and touchscreen and ambiences for displaying interactive 2D and 3D holograms, and in and stored among storage memories

16. The method according to claim 14 whereby said interactive digital display panels comprising the processes of:
individual approve functions attaching to and submitting said chronologically ordered relational health and healthcare data into and shared among said storage memories; and
individual delete functions attaching to and deleting individual health and healthcare data.
